(19) United States
(12) Patent Application Publication
(10) Pub. No.: US 2026/0216388 A1
(43) Pub. Date: Jul. 30, 2026

(54) DRAINPIPE BIOCIDE DELIVERY APPARATUS

(71) Applicants: Zoltan Kurucz, Boca Raton, FL (US); Pal Kurucz, Coconut Creek, FL (US)

(72) Inventors: Zoltan Kurucz, Boca Raton, FL (US); Pal Kurucz, Coconut Creek, FL (US)

(21) Appl. No.: 19/441,655

(22) Filed: Jan. 6, 2026

Related U.S. Application Data
(60) Provisional application No. 63/742,071, filed on Jan. 6, 2025.

(51) Int. Cl. A61L 2/18; A61L 2/26

(52) U.S. Cl. CPC A61L 2/18; A61L 2/26; A61L 2202/15

(57) ABSTRACT

A biocide delivery apparatus for a drainpipe system has an in-line central chamber with a dissolution well for retaining stagnant water when there is no fluid flow through the pipe system. It is connected in-line to a drainpipe or other pipe system by an inlet and an outlet. An upper housing extends upward from the central chamber and is sealed with an upper lid. A cartridge is removably inserted into the upper housing and extends down to the bottom of the well below the central, in-line chamber. One or more holes at the bottom of the cartridge allow a biocide block inside the cartridge to be exposed to stagnant water retained by the well. The central chamber may include guide walls that aligned with openings in the cartridge to facilitate turbulence and incoming water to better dissolve a biocide tablet.

### CROSS-REFERENCE TO RELATED APPLICATIONS

This application claims priority to U.S. Provisional Application Ser. No. 63/742,071 filed on Jan. 6, 2025.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

Not Applicable

### BACKGROUND OF THE INVENTION

### Field of the Invention

The present invention relates to an apparatus for delivering a biocide to a drainpipe or other pipe system. More particularly, the invention relates to a static, easily maintained in-line delivery system for delivering a biocide to prevent the buildup of biofilm in a drainage or other pipe system experiencing intermittent flow.

### Description of the Related Art

Many devices require drainage of one type or another. One particularly common system is a residential or commercial air-conditioning device. It is well-known that water precipitates on the coils of air conditioners. This water is collected in a drip pan and is carried by one or more drainpipes to the exterior of the building and the water is simply deposited onto the ground. In general, drainpipes have slow or intermittent flow of water through them. Unfortunately, warm, moist, shaded conditions are ideal for bacteria, algae, mold and other microorganisms that thrive in such environments. As a result, drainage systems are commonly plagued by the growth of these microorganisms. Many air-conditioning systems are now and equipped with a float in the drip pan to automatically shut off the system when the drainpipe is clogged, to prevent overflow of water from the drip pan and the resulting damage therefrom. This is inconvenient.

Routine maintenance is required to remove clogs of this organic material commonly referred to as biofilm or bioslime. Numerous devices and methods have been developed to more effectively remove or prevent the formation of biofilm in a drainage line, and/or to reduce the amount of maintenance required. Various pumping and/or suctioning devices have been developed to periodically treat a drainpipe system. It is also known to deliver a biocidal material to a drain system. However, because the actual flow through a drainage system can vary considerably, it is difficult to properly calibrate the delivery of a biocide to the system. If insufficient biocide is delivered, it does not prevent biofilm. If too much biocide is added, it can damage the pipe system itself and deposit unwanted chemicals into the environment.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide an apparatus and methods of use thereof for consistently administering an optimal amount of biocide to a pipe system.

### BRIEF SUMMARY OF THE INVENTION

Disclosed is a drainpipe biocide delivery apparatus having a central body, an inlet, an outlet, a lower well, an upper housing, a cartridge configured to extend down the housing, through the central body, and into the lower well, and one or more openings in the base of the cartridge.

In one embodiment, A drainpipe biocide delivery apparatus has a central chamber having an inlet, an outlet, and a dissolution well defined by a bottom of the central chamber, an an upper housing having a top opening. An internal cartridge has a plurality of openings at its bottom and configured to extend down through the housing, into the central body, and at least partially into the dissolution well. A removable cap covers the top opening of the upper housing, and the cartridge is configured to contain one or more chlorine tablets at its bottom. The cartridge optionally includes a flange secured between the top opening of the upper housing and the cap, and the upper housing and the cartridge may be at least semi-transparent.

In another embodiment, the drainpipe biocide delivery apparatus further comprising an inlet adapter and an outlet adapter that allow the drainpipe biocide delivery apparatus to rotate about an axis defined by the inlet and the outlet. The upper housing may include a base that extends in an upstream direction and in a downstream connection and is removably attached to the central chamber.

In another embodiment, the base of the upper housing has an upstream guide pin inserted into a radial slot on the inlet adapter, and a downstream guide pin inserted into a radial slot on the outlet adapter.

In another embodiment, the bottom of the cartridge has four equally spaced lateral openings separated by four equally spaced munions and a central bottom opening. The drainpipe biocide delivery apparatus has a central chamber with two upstream diagonal guide walls and two downstream diagonal walls that each abut one of the munions of the cartridge, thereby creating two side convection wells, each between one of the upstream diagonal walls and one of the downstream diagonal walls. The cartridge may optionally abut the bottom of the dissolution well. The inlet and the outlet each include o-rings to form a seal. The cap may similarly include an internal O-ring that abuts against the flange of the cartridge, and the cap is secured to the top of the upper housing by a bayonet lock.

It is therefore an object of the present invention to provide an apparatus and methods of use thereof to prevent the formation an accumulation of biofilm or other materials in a pipe system.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a cross-sectional view of a biocide delivery apparatus in accordance with the principles of the invention;

FIG. 2 is a side elevation view of an alternative embodiment of a biocide delivery apparatus in accordance with the principles of the invention;

FIG. 3 is a side view of an in-line chamber for an alternative embodiment of a biocide delivery apparatus in accordance with the principles of the invention;

FIG. 4 is a top plan view of an in-line chamber for an alternative embodiment of a biocide delivery apparatus in accordance with principles of the invention;

FIG. 5 is a bottom plan view of an in-line chamber for an alternative embodiment of a biocide delivery apparatus in accordance with principles of the invention;

FIG. 6 is a side elevation view of an upper housing for an alternative embodiment of a biocide delivery apparatus in accordance principles of the invention;

FIG. 7 is a perspective view of an an upper housing for an alternative embodiment of a biocide delivery apparatus in accordance with principles of the invention;

FIG. 8 is a side elevation view of a cartridge for an alternative embodiment of a biocide delivery apparatus in accordance with principles of the invention;

FIG. 9 is a side perspective view of a cartridge for an alternative embodiment of a biocide delivery apparatus in accordance with principles of the invention;

FIG. 10 is a partially exploded view of an alternative embodiment of a biocide delivery apparatus in accordance with principles of the invention;

FIG. 11 is a side elevation view of an alternative embodiment of a biocide delivery apparatus in accordance with principles of the invention;

FIG. 12 is a side elevation view of a completely assembled alternative embodiment of a biocide delivery apparatus in accordance principles of the invention;

FIG. 13 is a side elevation view of an alternative embodiment of an upper housing for a biocide delivery apparatus in accordance with principles of the invention;

FIG. 14 is another side elevation view of an alternative embodiment of an upper housing for a biocide delivery apparatus in accordance principles of the invention;

FIG. 15 is a top plan view of another alternative embodiment of a biocide delivery apparatus in accordance with principles of the invention;

FIG. 16 is a side cross-sectional view of another alternative embodiment of a biocide delivery apparatus in accordance principles of the invention;

FIG. 17 is a cross-sectional view of a adapter for a biocide delivery apparatus in accordance with principles of the invention;

FIG. 18 is a perspective view of a adapter for a biocide delivery apparatus in accordance with principles of the invention;

FIG. 19 is a side elevation view of a biocide delivery apparatus with a adapter in accordance with principles of the invention;

FIG. 20 is a top plan view of a biocide delivery apparatus with a adapter in accordance with principles of the invention;

FIG. 21 is another top plan view of a adapter for a biocide delivery apparatus in accordance with the principles of the invention;

FIG. 22 is an environmental view of a biocide delivery apparatus with adapters in accordance with principles of the invention;

FIG. 23 is an environmental view of a partially rotated biocide delivery apparatus in accordance principles of the invention;

FIG. 24 is an environmental view of a fully rotated biocide delivery apparatus in accordance with principles of the invention;

FIG. 25 is a cross-sectional view of another alternative embodiment of a biocide delivery apparatus in accordance with principles of the invention;

FIG. 26 is a front perspective view of another alternative embodiment of a biocide delivery apparatus in accordance with principles of the invention;

FIG. 27 is a top plan view of another alternative embodiment of a biocide delivery apparatus in accordance with principles of the invention;

FIG. 28 is a perspective view of an upper housing for a biocide delivery apparatus in accordance with principles of the invention;

FIG. 29 is a side cross-sectional view of a cap for a biocide delivery apparatus in accordance with principles of the invention;

FIG. 30 is a perspective view of a central chamber for a biocide delivery apparatus in accordance with the principles of the invention;

FIG. 31 is a perspective view of a cartridge for a biocide delivery apparatus in accordance with principles of the invention;

FIG. 32 is a top perspective view of a central chamber for a biocide delivery apparatus in accordance with the principles of the invention;

FIG. 33 is a partially assembled perspective view of an upper housing, a, and a cartridge for a biocide delivery apparatus in accordance with principles of the invention;

FIG. 34 is a perspective view of a cartridge been inserted into a central chamber of a biocide delivery apparatus in accordance with principles of the invention;

FIG. 35 is a top cross-sectional view of a central chamber of a biocide delivery apparatus in accordance with principles of the invention;

FIG. 36 is a side cross-sectional view of a central chamber of a biocide delivery apparatus in accordance with principles of the invention.

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices are shown in block diagram form in order to facilitate describing the various embodiments herein. Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

Unless otherwise indicated, all numbers expressing quantities of ingredients, dimensions, reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

The embodiments of the invention disclosed here in her described primarily in relation to exemplary drainpipes affiliated with air-conditioning systems such as drainpipes leading from a drip pan. Those skilled in the art will appreciate that the present invention may be applied to a wide variety of drainage systems and also may be useful for any of numerous piping systems where it is desirable to prevent the aggregation, glute nation were forming a biological material which may lead to clogging of the piping system or other deleterious, unwanted results. The present invention is useful in commercial settings, residential settings, as well as maritime and Marine settings, and may be used in any of a wide variety of vehicles from automobiles to airplanes.

Various embodiments of the disclosure could also include permutations of the various elements recited in the claims as if each dependent claim was a multiple dependent claim incorporating the limitations of each of the preceding dependent claims as well as the independent claims. That is, the combinations of the various components of the invention are not limited to those combinations expressly shown in the Figures. Unless expressly stated otherwise, components described in one embodiment may be interchanged with components of the same name found in other embodiments. Such permutations are expressly within the scope of this disclosure.

Disclosed is a biocide delivery apparatus that may be retrofitted in line to existing drainpipes or other conduits and is particularly suited for systems that experience intermittent flows. The apparatus has a central chamber in fluid communication with an inlet and an outlet that allow it to be inserted in line with existing conduit systems. At the bottom of the chamber is a dissolution well. An upper housing extends upward from the chamber and is aligned with the well. A removable at the top of the upper housing allows a cartridge to be inserted into the device. The bottom of the cartridge is positioned inside the well. One or more openings in the cartridge our positions at a level inside the well and substantially below the bottoms of the inlet and outlet. A dissolvable biocide is placed within the cartridge. The dissolvable biocide does not dissolve immediately and water. However, the dissolvable biocide dissolves in water trapped within the well when there is no flow through the system. As a result, the water within the well has a high concentration of the biocide. As water flows through the system, it mixes with the water in the well. As a result, water exiting and flowing downstream of the delivery apparatus includes dissolved biocide, thereby preventing the growth and accumulation of biofilm. Because biocide is only provided downstream from the delivery apparatus, it may be desirable to position the delivery apparatus as far upstream as is practical. For example, for an air-conditioning drip pan drain pipe, it may be desirable to position the delivery apparatus between the drip pan and drain pipe.

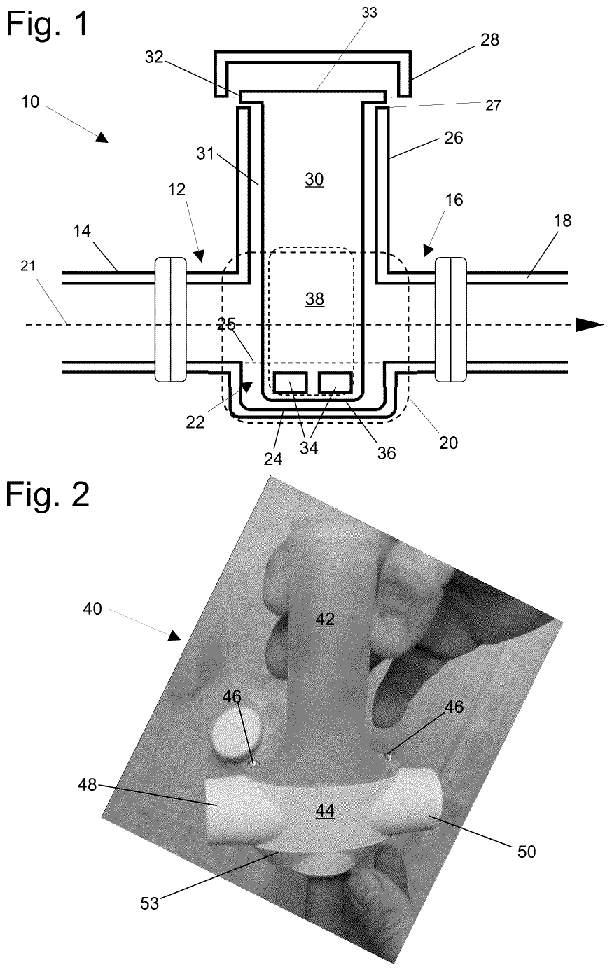

FIG. 1 shows an exemplary embodiment of a biocide delivery apparatus 10 in accordance with the principles of the invention. The device 10 has an inlet 12 connected to an upstream drainpipe 14 and an outlet 16 connected to a downstream drainpipe 18 of a drainpipe assembly. The central chamber 20 of the apparatus 10 is configured to lie in-line with the drainpipe system into which it is incorporated. That is, a longitudinal direction 21 may be defined by the inlet 12 and outlet 16, and is in line with the direction of fluid flow through the apparatus 10.

The in-line central chamber 20 of this embodiment has substantially the same cross-sectional dimensions as the inlet 12 and outlet 16 connected to the upstream and downstream pipes 14 and 18, respectively, of a drainpipe system. A dissolution well 22 extends downward to a bottom 24 of the central chamber 20. The top 25 of the dissolution well 22 is defined as the bottom of the central chamber 20 and in this embodiment is also defined by the bottoms of the inlet 12 and outlet 16. An upper housing 26 extends upward from the central chamber 20 and is accessible by removing the cap 28. When the cap 28 is removed, a cartridge 30 may be removably inserted through the top 27 of the upper housing 26.

The cartridge 30 of this embodiment is substantially cylindrical and is defined by a cylindrical wall 31 extending from a horizontal flange 32 at the top 33 of the cartridge 30 to the its bottom 36. The flange 32 rests on the top 27 of the upper housing 26 when it is inserted. The cartridge 30 extends downward into the dissolution well 22 such that the lateral openings 34 of the cartridge 30 are positioned below the top 25 of the dissolution well 22. When the cap 28 is attached to the top of the upper housing 26, it secures the cartridge 30 in place by sandwiching the flange 32 and between the cap 28 and the top 27 of the housing 26. In this embodiment, the bottom 36 of the cartridge 30 does not abut the well bottom 24 such that there is a gap between the bottom of the cartridge 30 and the well bottom 24. The cartridge 30 may optionally include one or more bottom openings on the cartridge's bottom 36. The cartridge 30 also includes one or more solid or semisolid blocks 38 of a soluble biocide. The biocide block 38 may be formulated for a specific rate of dissolution depending on the type of pipe system, temperature, flow rate of the pipe system and other factors known in the art.

When water or other liquid flows through the biocide delivery apparatus 10, liquid will collect in the dissolution well 22 such that there is always water in the well even when there is no liquid flowing through the drainpipe system 11. The stagnant water remaining in the dissolution well 22 when flow has stopped, remains in contact with the biocide block, continuing to dissolve it. As a result, the stagnant water in the well between periods of flow through the pipe system acquires a high concentration of biocide. Those skilled in the art will appreciate that some tablets of solid biocide dissolves relatively slowly, insufficient biocide may not dissolve sufficiently into running water passing over it. The dissolution well 22 keeps at least a portion of the tablet submerged, allowing it to slowly dissolve to a relatively high concentration of biocide. This concentration is higher than necessary to keep the drainpipe system free of biofilm and the like. When water flows through the system, it mixes with the highly concentrated well water, diluting its to a lower concentration still sufficient to kill microorganisms and prevent biofilm, but low enough to not damage or deteriorate the pipe system itself. Drainage pipe systems often utilize PVC pipe, rubber tubing, and other relatively inexpensive materials that over time can be damaged by strong chemicals. It is also bad for the environment to dump high concentrations of biocidal chemicals into the ground from a drainpipe. Thus, the dissolution well 22 may optionally be lined with a more durable material than PVC or other plastics. The dissolution well 22 of the invention thus provides a static system for generating a sufficient concentration of biocide to treat the downstream drainpipe system when water is flowing through the apparatus 10, while also allowing the drainage water to be calm sufficiently diluted prior to its exit from the drainpipe system.

Often, flow of liquid through a drainpipe system or other pipe system is relatively slow, low volume and low pressure. As a result, water flowing through a pipe may simply flow over a standing puddle such as the stagnant water in the well 22, without substantially mixing. To prevent this, the apparatus 10 in this embodiment includes a cartridge 30 which extends vertically through the central chamber 20, substantially in-line with the inlet 14 and outlet 18. The presence of the cartridge 30 redirects fluid flow into the dissolution well 22 and around and underneath the cartridge 30. This not only forces downstream flowing water to intersect directly with the stagnant concentrated water, and also creates turbulence to more effectively mix the stagnant and flowing water together and “flushes” the well 22. Thus, the present invention ensures that the concentrated stagnant water in the well 22 flows downstream effectively.

FIGS. 2 to 12 show an alternative embodiment of a biocide delivery apparatus 40 in accordance with principles of the invention. In this embodiment, the upper housing 42 is semi-transparent and removably attachable to the central chamber 44 by a pair of screws 46. An inlet 48 and outlet 50 extend from opposing sides of the chamber 44. A well 52 extends downward from the in-line chamber 44. The top 53 of the well is again defined by the common bottoms of the chamber 44, inlet 48 and outlet 50. This is the region in which water stagnates when there is no flow through the system to which the apparatus 40 is attached. Those skilled in the art will appreciate that there are a variety of methods for attaching the apparatus 40 in line with a pipe system via inlet 48 and outlet 50. Both apparatus 10 and apparatus 40 show inlets and outlets positioned on opposing sides of the central in-line chamber. The in-line central chamber 44 of this embodiment, however, has a cross-sectional area that is slightly larger than the cross-sectional area of the inlet and outlet. This additional girth accommodates a cartridge 54, shown below. Because the upper housing 42 is semi transparent, an operator is able to discern, by inspection alone, whether the cartridge is running low on biocide.

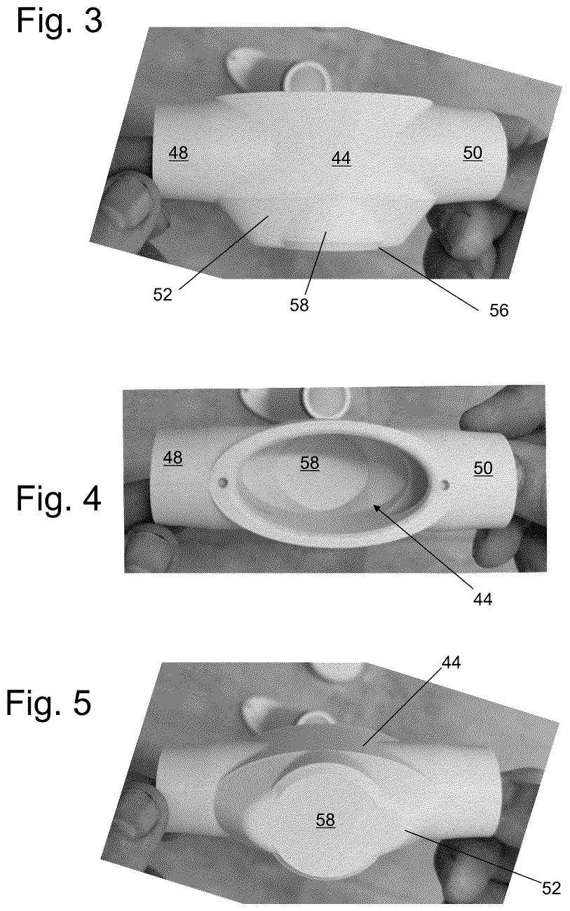

FIGS. 3 to 5 show the in-line chamber 44 with the upper housing 42 removed. The bottom 56 of the well 52 includes a circular, central seat 58 complementary to the bottom of the cartridge 54. The in-line chamber 44 itself has an oval horizontal cross-section that forms extra volume upstream and downstream of the cartridge 54 when it is inserted. This further improves mixing by creating turbulence and also allows water to accumulate in the event of a sudden surge of water entering through the inlet 48. Buildup of water in this additional volume upstream of the cartridge 54 also serves to push incoming water down through the well 52. Additional volume is created in the chamber both upstream and downstream of the location of the cartridge so that the apparatus 40 is symmetric and cannot be inserted a wrong way. This simplifies installation of the apparatus 40. The transverse width of the in-line central chamber 44 is sized to accommodate the cartridge 54 and does not add significant volume in a transverse direction.

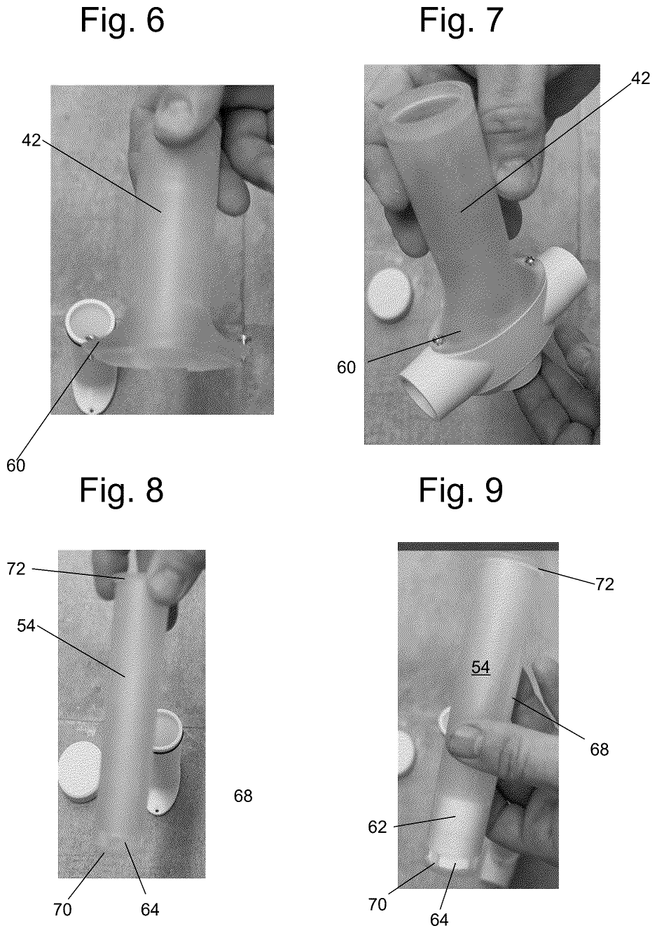

FIGS. 6 and 7 show the upper housing 42 in more detail. The bottom 60 of the housing 42 flares outward on two opposing upstream and downstream sides. Because the housing 42 is semi transparent, an operator is able to readily determine whether there is debris or other material creating blockage in the apparatus 40. Because the housing 42 is removable, removing blockage or other maintenance on the apparatus 40 may be performed simply by removing the housing 42, rather than entirely disconnecting the apparatus 40.

FIGS. 8 and 9 show the cartridge 54 for use with the biocide delivery apparatus 40. Cartridge 54, like housing 42, is semi-transparent. As can be seen in the Figures, an operator can readily identify whether a biocide block 62 is present within the cartridge 54. Thus, simple visual inspection allows an operator to determine whether additional biocide must be added to the apparatus. The openings 64 at the bottom of the outer wall 68 are relatively large to maximize the surface area of the biocide exposed to water stagnating within the well 52. Those skilled in the art will appreciate that the greater surface area exposed to the water, the more effective dissolution will be. The cartridge 54 may optionally include means for adjusting the size of the openings 64. The outer wall 68 extends from the bottom 70 to the upper annular flange 72 of the cartridge 54.

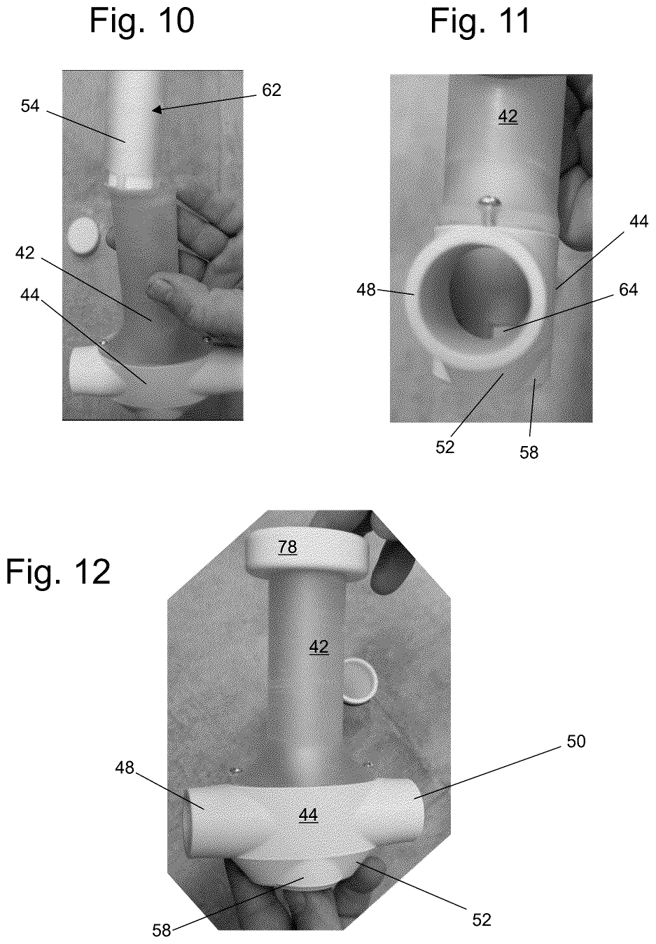

FIGS. 10 to 12 show the assembly of the apparatus 40. After biocide is inserted into the cartridge 54, it is slid down the housing 42 until the bottom 70 of the cartridge 54 abuts against the seat 58 in the bottom of the well 52. The cap 78 is then secured to the top of the housing 42, securing the cartridge within the apparatus 40. Once the apparatus 40 is inserted into a drain pipe system, the only maintenance required his occasional visual inspection. When the cartridge is low of biocide, the cap 78 is removed so that biocide may be added to the cartridge 54 it is not necessary to remove the cartridge 54 when inserting additional biocide, but doing so allows for more thorough inspection.

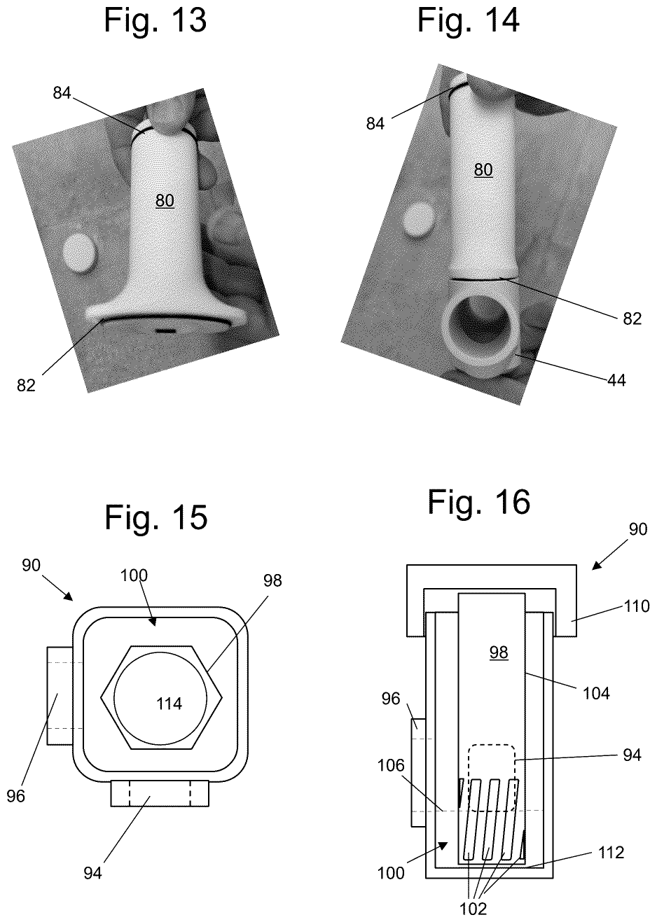

FIGS. 13 and 14 show an alternative embodiment of an upper housing 80 configured for use with biocide delivery apparatus 40 above. Upper housing 80 is completely opaque, thus an operator cannot visually inspect the interior of an apparatus when upper housing 80 is used. Upper housing 80 includes to gaskets, O ring 82 which seals the connection between the upper housing 80 and a central chamber, for example in-line chamber 44. O ring 84 seals the connection between the upper housing 80 and a lid. Those skilled in the art will appreciate that these O-ring gaskets may be adapted for use with upper housing 42 shown above or other upper housings.

FIGS. 15 and 16 show another alternative embodiment of a biocide delivery apparatus 90 in accordance with principles of the invention. In this embodiment, the central in-line chamber 92 is substantially rectangular and the inlet 94 and outlet 96 are perpendicular, not on opposing sides. Nonetheless, those skilled in the art will appreciate that the inlet 94 and outlet 96 defined in a direction of fluid flow. Inlet 94 and outlet 96 also have a substantially rectangular cross-sections. In addition, the cartridge 98 has a hexagonal cross-section and is substantially smaller than the interior of the chamber 92. This configuration creates additional turbulence to better mix stagnant water in the bottom of the well 100 with water entering through the inlets 94. In addition, cartridge 98 has a plurality of diagonal rectangular openings 102 in the lower region of its sidewall 104. The configuration of these openings further encourages turbulence in the well to improve mixing. The rectangular openings 102 of cartridge 98 also extend substantially above the top 106 of the well 100. Cartridge 98 has no annular flange on its top, but is instead held in place by a friction fit between the cap 110 and the bottom 112 of the well 100. Those skilled in the art will appreciate that one or more of these variations and design and configuration may be adapted to other embodiments. Biocide block 114 is also different from the biocide blocks of previous embodiments. Specifically it consists of a highly concentrated liquid contained within an osmotic bag. Highly concentrated biocide may be provided in the form of a gel, semisolid or highly viscous material. Osmotic bags come in a wide variety of gauges that may be used to control the rate of osmotic flow. It may be desirable in some environments to use a system such as this rather than a simple block of dissolvable biocide material.

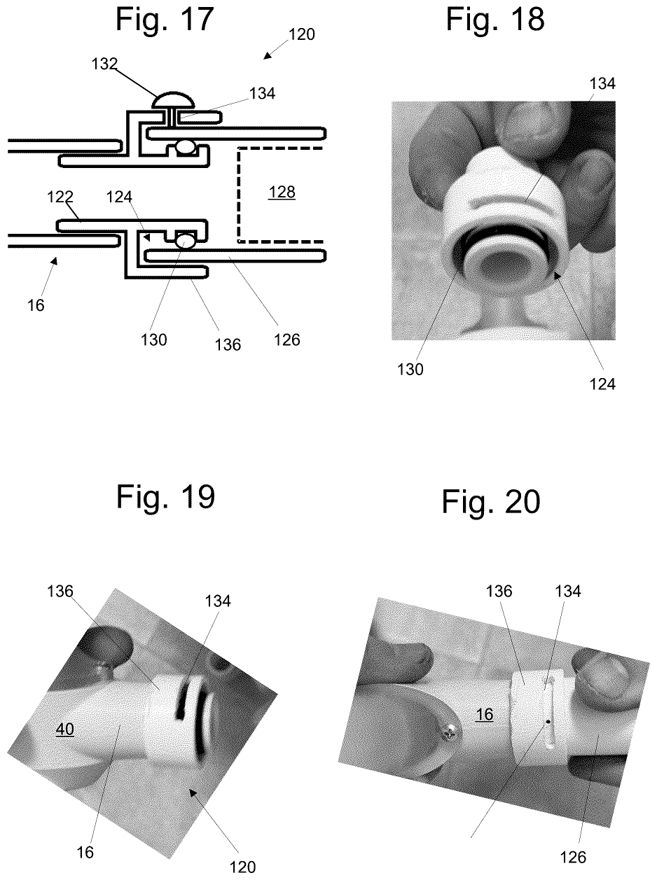

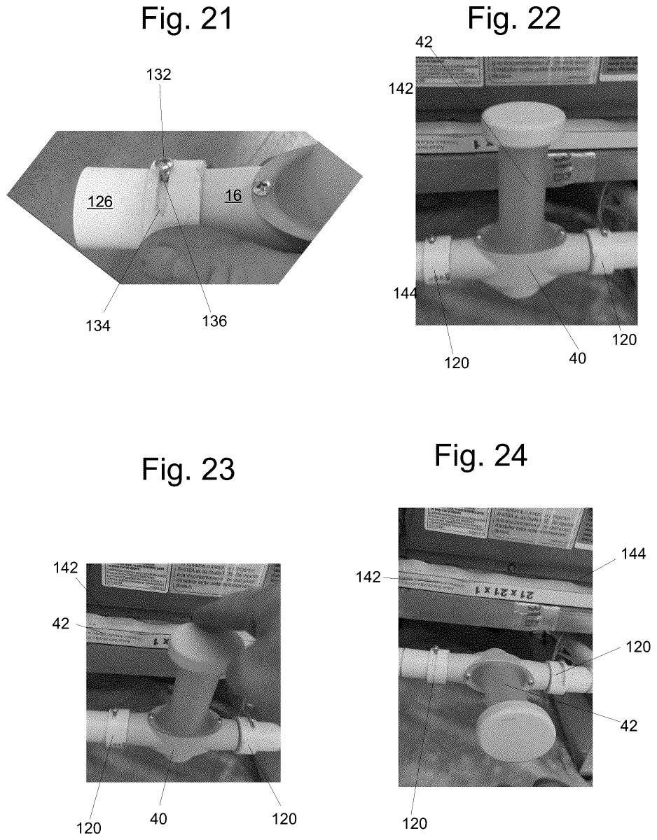

FIGS. 17 to 24 show a rotatable adapter 120 that may optionally be used attached to the inlet and outlet of a biocide delivery apparatus when retrofitting an existing drainpipe system. Adapters 120 are attached to both the inlet and outlet of a biocide delivery device to allow it to be rotated to allow access to adjacent devices. The rotatable adapter 120 is attached to an outlet 16 of delivery apparatus 40 shown in FIGS. 2 to 12. The adapter 120 has a cylindrical insertable flange 122 extending in a first direction and configured to be inserted into the outlet 16. The insertable flange 122 may attach to the interior wall of the outlet 16 using any of a variety of means from PVC glue to a simple friction fit. Typically, because drainpipe systems are usually low pressure, a friction fit will often suffice. Those skilled in the art will appreciate that the insertable flange 122 may optionally be replaced with an outer flange extending around the outside of the outlet 16, a U-shaped channel into which the outlet 16 may be inserted or various other connecting mechanisms.

The rotatable adapter 120 also includes a U-shaped annular channel 124 extending in a direction opposite to the insertable flange 122, and configured to accept one end of a connecting socket 126 for attachment to the upstream end of a downstream drainpipe 128. The U-shaped channel 124 includes at least one gasket. In this embodiment, the gasket is an O-ring 130. The O-ring 130 prevents water from leaking out of the system and also provides sufficient frictional resistance to prevent free rotation of the socket 126 and the adapter 120 relative to each other, but still allowing rotation if sufficient torque is applied.

Once the socket 126 is inserted into the U shaped channel, a translating retention bolt or screw 132 inserted through a slit 134 in the outer wall 136 of the U-shaped annular channel 124. FIG. 18 shows the adapter 120 detached from the outlet 16 and socket 126. As may be seen in this Figure, slit 134 extends around the outer wall 136 approximately 90°.

FIGS. 19 to 22 illustrate how the adapter 120 is integrated into a drainpipe system with the in-line biocide delivery apparatus. First, the adapter 120 is attached to the delivery apparatus 40 by inserting the inner flange 122 into the outlet 16. Next, as shown in FIG. 20, the socket 126 is inserted into the U-shaped channel 124. The socket 126 includes a hole 138 for receiving the retention bolt or screw 132. This whole 138 in the socket 126 is aligned with slit 134. The head of the bolt or screw 132 is sized to be slightly larger than the slit 134, and secures the socket 126 in place within the U-shaped channel 124 while also allowing 90° rotation of the biocide delivery apparatus relative to a drainpipe system.

FIGS. 22 to 24 show the biocide delivery apparatus 40 inserted in line near the upstream end of a drainpipe system. It is desirable usually to put the apparatus 40 as far upstream as practical. However, the upstream region of a drainpipe system is often in close proximity to other components of an air-conditioning or other system. In FIGS. 22 to 24, the apparatus 40 is proximal to the slot 144 and air-conditioning filter 142. The upper housing 42 blocks access to the slot 144 and filter 142. But because the rotatable adapters 120 have been used to attach the apparatus 40 in line to the drainpipe system, the entire apparatus 40 may be rotated 90° by applying a torque force to the upper housing 42, as shown in FIG. 23. The apparatus 40 may be rotated until it is no longer blocking the slot 144, as shown in FIG. 24. Once the air-conditioning filter 142 is replaced, the apparatus 40 may be rotated in the opposite direction such that the upper housing 42 extends vertically upward again.

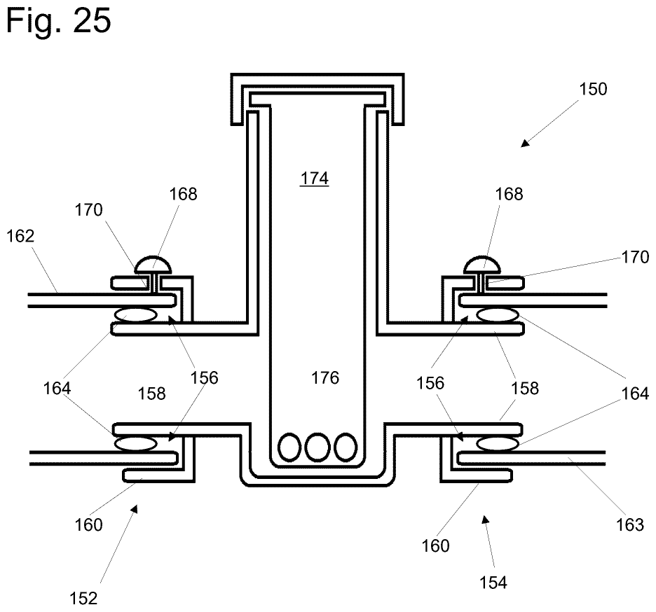

FIG. 25 shows another alternative embodiment of a biocide delivery apparatus 150. In this embodiment, the components of the adapter 120 have been Incorporated into the inlet 152 and outlet 154 of the apparatus 150 itself directly. A U-shaped annular channel 156 is formed by the cylindrical inner wall 158 and a cylindrical outer wall 160 that forms the inlet 152 and an outlet 154. The upstream pipe 162 extends into the channel 156 and a substantially watertight seal is formed by O-ring gasket 164. Similarly, the downstream pipe 163 is inserted into the outlet 154. A retention bolt 168 is attached to the upstream pipe 162 through a slit 170. The apparatus 150 also includes a cartridge 174 that has a plurality of circular holes 176 around its bottom region. This illustrates a another possible configuration for the openings in a cartridge. Those skilled in the art will appreciate that the modifications shown in FIG. 25 may be Incorporated into other embodiments of the invention, including those explicitly described above.

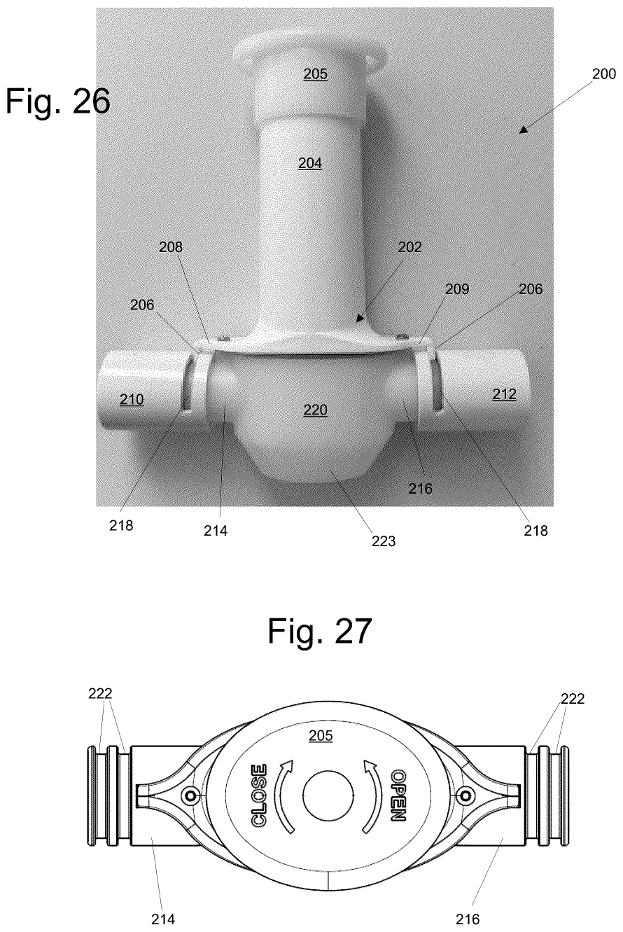

FIG. 26 shows another alternative embodiment of a drainpipe biocide delivery apparatus 200 in accordance with principles of the invention. In this embodiment, retention bolt are not used to facilitate rotating of the delivery apparatus 200. Instead, the base 202 of the upper housing 204 includes two downward pointing guide pins 206, one at the upstream end 208 of the base 202 and the other at the downstream 209 end of the base 202. As with the previous embodiment, the upstream and downstream adapters, 210 and 212 respectively. Upstream adapter 210 is slid over the inlet 214 while adapter 212 is slid over the outlet 216. The adapters are slid a distance to allow the guide pins 206 to align with slots 218. When the upper housing 204 is attached to the central chamber 220, the guide pins 206 insert into the slots 218, securing both adapters 210 in 212 in place so that they do not loosen and become disengaged. These guide pins 206 and slots 218, as with the previous embodiment, allow the apparatus 202 rotate in a plane transverse to the direction of fluid flow. The cap 205 is removably attached to the top of the upper housing 204. The bottom region 223 of the central chamber 220 tapers inward and forms the dissolution well as explained in more detail below. FIG. 27 shows the biocide delivery apparatus 200 with the adapters 210 and 212 removed. The inlet 214 and outlet 216 each include two channels 222 for O-rings in order to provide a tight seal when the adapters are slid over them. The cap 205 has an elliptical shape that is more ergonomic for turning the cap 205 to either attach or remove it.

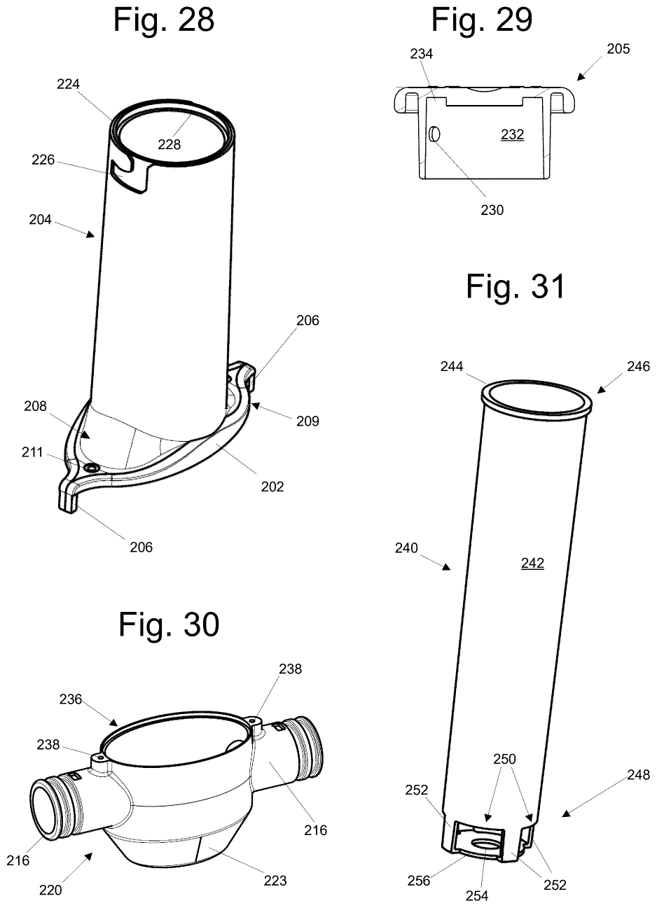

FIGS. 28 to 31 shows the components of apparatus 200 individually and in more detail. FIG. 28 shows the upper housing 204 with the upstream 208 and downstream 209 extensions of the upper housing's base 202. The top 224 of the upper housing 204 includes a female component of a bayonet lock 226 on its exterior and a shoulder 228 just inside the top 224. In this embodiment, the upstream and downstream extensions 208 and 209 also include screw holes 211 for attaching the upper housing 204 to the central chamber 220.

FIG. 29 shows male component of a bayonet lock 230 on the interior wall 232 of the cap 205. The cap 205 also includes an annular channel 234 at the top of the inner wall 232 which may optionally be configured as a seat for an O-ring. Use of an O-ring may form a tighter seal and may also create an upward bias in the cap 205 to more securely and tightly engage the bayonet lock.

FIG. 30 shows the central chamber 220. It has a substantially elliptical top opening 236 which is complementary to the base 202 of the upper housing 204. Two screw posts 238 correspond to the screw holes 211 in the upper housing's base 202.

FIG. 31 shows the cartridge 240 for the biocide delivery apparatus 200. The cartridge 240 is defined by an elongate side wall 242 extending from a flange 244 at its top 246 down to its bottom 248. At the bottom 248 of the cartridge 240 is a series of equally sized and spaced openings 250 separated by equally sized and spaced munions 252 that connects the cartridge 242 the bottom tablet tray 254 that itself has a central opening 256. The flange 244 is configured to rest on the shoulder 228 of the upper housing 204 when it is inserted. The 205 is configured to abut the top 246 of the cartridge 240 such that the cartridge 240 is held rigidly in place within the cap 205 is attached to the upper housing 204.

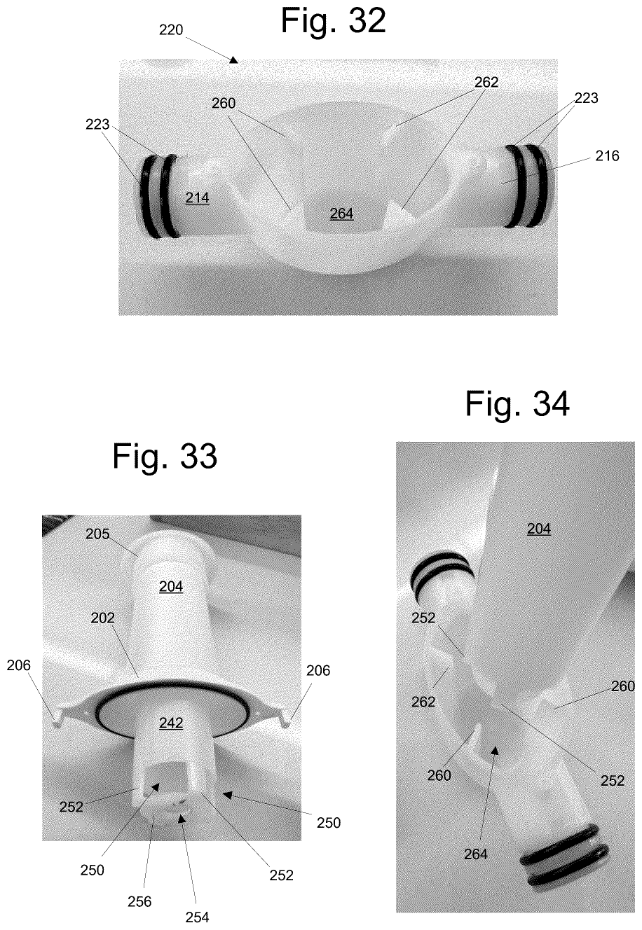

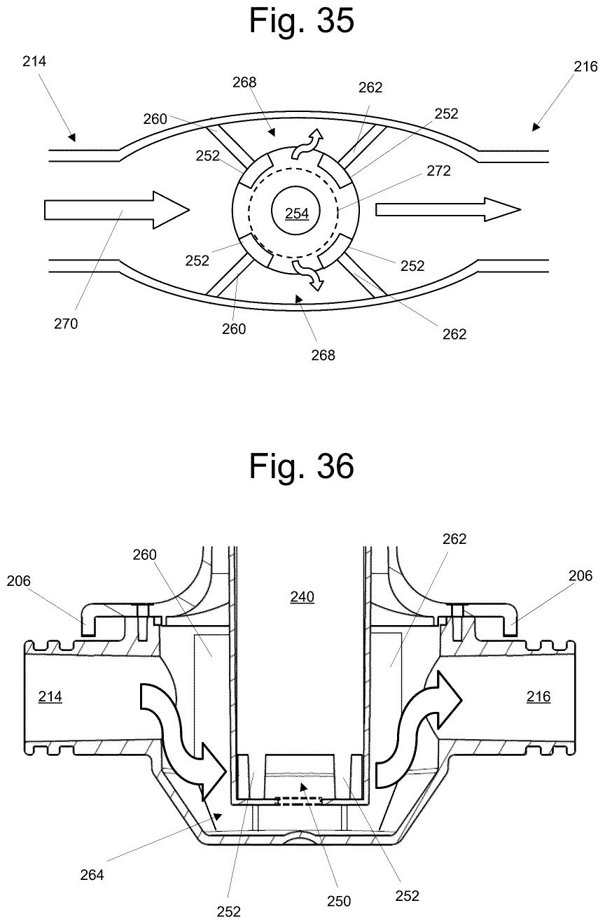

Referring to FIG. 32, the interior of the central chamber 220 is elliptical in shape and includes four diagonal guide walls. The guide walls adjacent to the inlet 214 are referred to as the two upstream guide walls 260 while the guide walls adjacent to the outlet 216 are referred to as the downstream guide walls 262. In this embodiment, the central chamber 220 is symmetric and therefore identifying upstream and downstream, and inlet and outlet, is trivial and they may be interchanged. These guide walls 260 and 262 hold the cartridge 240 securely in place and also create two sidewells as shown in FIG. 35 below. The guide walls 260 and 262 extend upward through the dissolution well 264. As explained in more detail below, the formation of the sidewalls 264 by the guide walls 260 and 262 improves dissolution and overall performance of the apparatus. FIG. 32 also shows O-rings 223 within the O-ring seats 222. FIG. 33 shows the bottom of the base 202 of the upper housing 204. The O-ring 268 helps form a tight seal with the central chamber 220. The cartridge 240 in this Figure is secured within the upper housing 204 because the cap 205 is secured in place. FIG. 34 shows how the munions 252 align with the diagonal guide walls 260 and 262 as it is inserted into the central chamber 220. Those skilled in the art will appreciate that an elongate structure such as the cartridge 204, even though it is secured at its top end, may “wobble” at its bottom ends, creating undue stress on the components of the apparatus 200 and shortening the lifespan of those components. The diagonal guide walls 260 and 262, by abutting against the munions 252, rigidly secures the cartridge 204 to prevent wobble.

FIGS. 35 and 36 show the interior of the central chamber 220 of the assembled apparatus 200. The two upstream guide walls 260 abut two of the munions 252, while the downstream guide walls 262 abut the other munions 252. This forces incoming fluid 270 into the dissolution well 264 and/or through the bottom of the cartridge 240. Fluid entering the cartridge 240 experiences turbulence and some of it flows into the side wells 268. The turbulence assists in the dissolution of the biocide tablet 272. Fluid entering the side wells 268 may then flowed downward to the bottom of the dissolution well 264 and then out through the outlet 216. The diagonal guide walls also prevent in flowing fluid 270 from circumventing the dissolution well 264 and the cartridge 240 and simply flowing around the cartridge 240 and out of the outlet without substantially mixing with fluid in the dissolution well. Because fluid flowing into the side wells 268 experienced turbulence and flow more slowly into the dissolution well 264, more biocide dissolves into the fluid. This improves dissolution of the biocide when liquid in the dissolution well has not had sufficient time to adequately dissolved biocide and or when there is relatively constant fluid flow for a prolonged period.

Whereas, the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods and systems for carrying out the several purposes of the present invention. Descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. A drainpipe biocide delivery apparatus comprising:
a central chamber having an inlet, an outlet, and a dissolution well defined by a bottom of the central chamber;
an upper housing having a top opening;
a cartridge having a plurality of openings at its bottom and configured to extend down through the housing, into the central body, and at least partially into the dissolution well; and,
a removable cap covering the top opening of the upper housing;
wherein the cartridge is configured to contain one or more chlorine tablets at its bottom.

2. The drainpipe biocide delivery apparatus of claim 1 wherein a top of the cartridge includes a flange secured between the top opening of the upper housing and the cap.

3. The drainpipe biocide delivery apparatus of claim 2 wherein the upper housing and the cartridge are at least semi-transparent.

4. The drainpipe biocide delivery apparatus of claim 3 further comprising an inlet adapter and an outlet adapter that allow the drainpipe biocide delivery apparatus to rotate about an axis defined by the inlet and the outlet.

5. The drainpipe biocide delivery apparatus of claim 4 wherein the upper housing has a base that extends in an upstream direction and in a downstream connection and is removably attached to the central chamber.

6. The drainpipe biocide delivery apparatus of claim 5 wherein the base of the upper housing has an upstream guide pin inserted into a radial slot on the inlet adapter, and a downstream guide pin inserted into a radial slot on the outlet adapter.

7. The drainpipe biocide delivery apparatus of claim 6 wherein a bottom of the cartridge has four equally spaced lateral openings separated by four equally spaced munions and a central bottom opening.

8. The drainpipe biocide delivery apparatus of claim 7 wherein the central chamber further comprises two upstream diagonal guide walls and two downstream diagonal walls that each abut one of the munions of the cartridge, thereby creating two side convection wells, each between one of the upstream diagonal walls and one of the downstream diagonal walls.

9. The drainpipe biocide delivery apparatus of claim 8 wherein the bottom of the cartridge does not abut the bottom of the dissolution well.

10. The drainpipe biocide delivery apparatus of claim 9 wherein the inlet and the outlet each include at least one o-ring on an exterior side.

11. The drainpipe biocide delivery apparatus of claim 10 wherein the cap includes an internal o-ring that abuts against the flange of the cartridge.

12. The drainpipe biocide delivery apparatus of claim 11 wherein the cap is secured to the top of the upper housing by a bayonet lock.
